(19) United States
(12) Patent Application Publication
(10) Pub. No.: US 2009/0163513 A1
(43) Pub. Date: Jun. 25, 2009

(54) PROCESS FOR ZIPRASIDONE USING NOVEL INTERMEDIATES

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Itiyala Srinivas Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 10/596,675

(22) PCT Filed: Jan. 27, 2005
(86) PCT No.: PCT/IN05/00030

§ 371 Date: Jun. 21, 2006

(30) Foreign Application Priority Data
Jan. 27, 2005 (IN) PCT/IN05/00030

(51) Int. Cl. A61K 31/501; C07D 417/14; C07F 7/10

(52) U.S. Cl. 514/254.02; 544/368; 544/229

(57) ABSTRACT

The present invention relates to a novel process for the preparation of high purity ziprasidone and pharmaceutically acceptable acid addition salts of ziprasidone; and solvates and hydrates thereof using novel intermediates and a purification method for ziprasidone and pharmaceutically acceptable acid addition salts of ziprasidone; and solvates and hydrates thereof.

Thus, 1-(1,2-benzisothiazol-3-yl)piperazine is silylated with trimethylsilylchloride in methylene chloride in the presence of triethylamine and the solvent is distilled off to obtain silylated 1-(1,2-benzisothiazol-3-yl)piperazine. The silylated compound is reacted with 5-(2-chloroethyl)-6-chloro-oxindole in the presence of sodium carbonate to obtain ziprasidone.

### FIELD OF THE INVENTION

The present invention relates to a novel process for the preparation of high purity ziprasidone and pharmaceutically acceptable acid addition salts of ziprasidone; and solvates and hydrates thereof using novel intermediates and a purification method for ziprasidone and pharmaceutically acceptable acid addition salts of ziprasidone; and solvates and hydrates thereof.

### BACKGROUND OF THE INVENTION

### Ziprasidone of Formula (I)

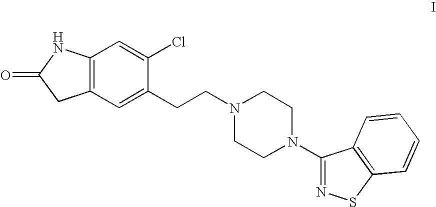

or 5-[2-[4-(1,2-Benzisothiazol-3-yl)-1-piperazinyl]ethyl]-6-chloro-1,3-dihydro-2H-indol-2-one and its salts are antipsychotic agents. Ziprasidone hydrochloride and related compounds and their therapeutic uses were disclosed in U.S. Pat. No. 4,831,031, which is hereby incorporated by reference. According to U.S. Pat. No. 4,831,031, ziprasidone can be prepared by reacting 1-(1,2-benzisothiazol-3-yl)piperazine and 5-(2-chloroethyl)-6-chloro-oxindole in a polar solvent, such as a lower alcohol, dimethylformamide or methylisobutyl ketone in the presence of a weak base.

U.S. Pat. No. 5,206,366 and U.S. Pat. No. 5,338,846 are described a process for preparing ziprasidone by reacting 1-(1,2-benzisothiazol-3-yl)piperazine with 5-(2-chloroethyl)-6-chloro-oxindole in water with a neutralizing agent such as sodium carbonate under reflux.

U.S. Pat. No. 6,150,366 is related to particle size distribution of ziprasidone or ziprasidone hydrochloride.

According to J. Med. Chem. 1996, 39, 143-148, ziprasidone is prepared by reacting 1-(1,2-benzisothiazol-3-yl)piperazine with 5-(2-bromoethyl)-6-chloro-oxindole in isoamyl alcohol solvent in the presence of sodium carbonate.

The publication No. US 2004/0152711 A1 is related to polymorphs of ziprasidone (amorphous ziprasidone hydrochloride and crystalline ziprasidone free base).

The publication No. WO 2004/089948 A1 is related to crystalline forms of ziprasidone hydrochloride monohydrate.

U.S. Pat. No. 5,359,068 is related to processes for the preparation of ziprasidone. Despite various processes disclosed in the prior art for the preparation of ziprasidone and salts thereof, still there is a need for producing ziprasidone and pharmaceutically acceptable acid addition salts of ziprasidone; and solvates and hydrates thereof in high purity.

One object of the present invention is to provide a process for preparing ziprasidone and pharmaceutically acceptable acid addition salts of ziprasidone; and solvates and hydrates thereof in high purity using novel intermediates.

Another object of the present invention is to provide a purification method of crude ziprasidone and pharmaceutically acceptable acid addition salts of ziprasidone; and solvates and hydrates thereof in high purity.

### SUMMARY OF THE INVENTION

The present invention provides a novel process to prepare 5-[2-[4-(1,2-Benzisothiazol-3-yl)-1-piperazinyl]ethyl]-6-chloro-1,3-dihydro-2H-indol-2-one (ziprasidone) of formula I:

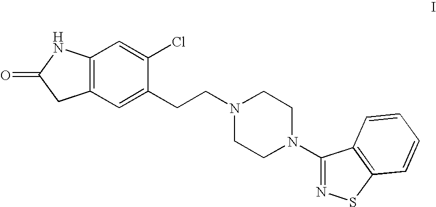

or a pharmaceutically acceptable salt thereof; or a solvate or a hydrate thereof; which comprises:
a) silylating 1-(1,2-benzisothiazol-3-yl)piperazine of formula II:

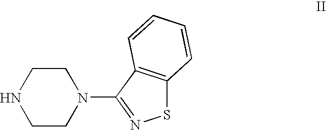

with a silylating agent to form the compound of formula III:

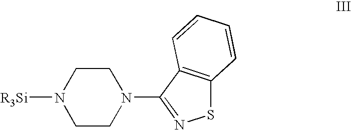

wherein R groups are independently alkyl;
b) reacting the silyl compound of formula III with 5-(2-haloethyl)-6-chloro-oxindole compound of formula IV:

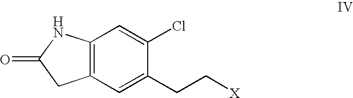

wherein X is fluoro, chloro, bromo or iodo;
in a solvent in the presence of a base to neutralize hydrohalic acid, at 40° C. to the reflux temperature of the solvent used to form the compound of formula I and optionally converting the compound of formula I into a pharmaceutically acceptable acid addition salt thereof; or a solvate or a hydrate thereof.

According to another aspect of the present invention there is provided another novel process for preparing ziprasidone and pharmaceutically acceptable acid addition salts of ziprasidone; and solvates and hydrates thereof. Thus 1-(1,2-benzisothiazol-3-yl)piperazine of formula II:

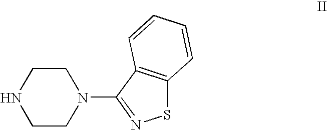

is reacted with 5-(2-haloethyl)-6-chloro-oxindole of formula IV:

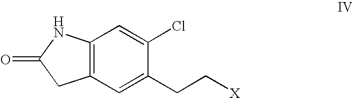

wherein X is fluoro, chloro, bromo or iodo;

in the presence of liquor ammonia and an alkaline metal carbonates such as sodium carbonate or potassium carbonate or an alkaline metal bicarbonate such as sodium bicarbonate or potassium bicarbonate to form ziprasidone of formula I and optionally converted ziprasidone formed into a pharmaceutically acceptable acid addition salts of ziprasidone; or solvate or a hydrate thereof.

According to another aspect of the present invention there is provided still another novel process for preparing ziprasidone and pharmaceutically acceptable acid addition salts of ziprasidone; and solvates and hydrates thereof.

Thus 1-(1,2-benzisothiazol-3-yl)piperazine of formula II:

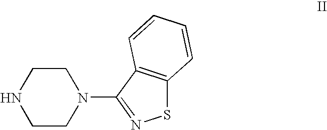

is reacted with 5-(2-haloethyl)-6-chloro-oxindole of formula IV:

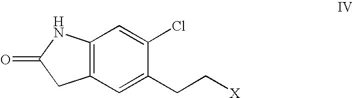

wherein X is fluoro, chloro, bromo or iodo;

in the presence of pyridine and aqueous monomethylamine to form ziprasidone of formula I and optionally converted ziprasidone formed into a pharmaceutically acceptable acid addition salts of ziprasidone; or a solvate or a hydrate thereof.

Present invention also provides a process for purification of ziprasidone which process comprises:

i) silylating crude ziprasidone of formula I:

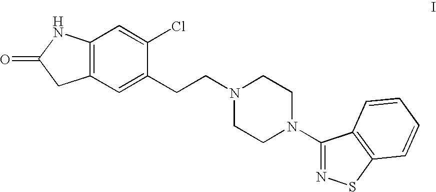

with a silylating agent to form silyl compound of formula V:

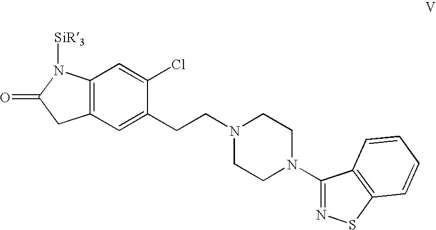

wherein R′ groups are independently alkyl, and

ii) deblocking the silyl protecting group of the compound of formula V formed in step (i) to precipitate ziprasidone of formula I as ziprasidone free base or a pharmaceutically acceptable acid addition salts; or a solvate or a hydrate thereof, as crystalline salt.

Silyl compounds of the formula V are novel and forms part of the invention.

### DETAILED DESCRIPTION OF THE INVENTION

According to one aspect of the present invention The present invention provides a novel process to prepare 5-[2-[4-(1,2-Benzisothiazol-3-yl)-1-piperazinyl]ethyl]-6-chloro-1,3-dihydro-2H-indol-2-one (ziprasidone) of formula I:

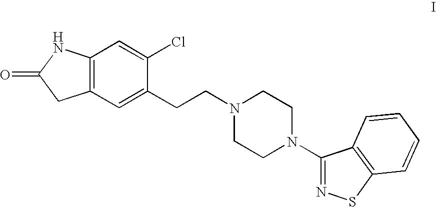

or a pharmaceutically acceptable salt thereof; or a solvate or a hydrate thereof; which comprises;
a) silylating 1-(1,2-benzisothiazol-3-yl)piperazine of formula II:

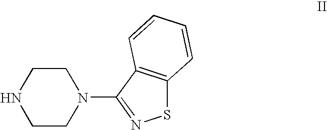

with a silylating agent to form compound of formula III:

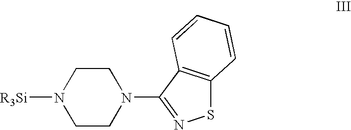

where in R groups are independently alkyl;
b) reacting the silyl compound of formula III with 5-(2-haloethyl)-6-chloro-oxindole compound of formula IV:

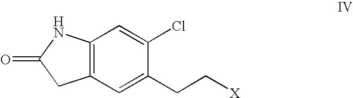

wherein X is fluoro, chloro, bromo or iodo;

in a solvent in the presence of a base to neutralize hydrohalic acid, at 40° C. to reflux temperature of the solvent used to form the compound of formula I and optionally converting the compound of formula I into a pharmaceutically acceptable acid addition salt thereof; or a solvate or a hydrate thereof.

Silylation can be performed by conventional method using conventional silylating agents.

Preferable silylating agents are selected from trialkylsilyl halides, N,O-bis(trimethylsilyl)-acetamide and N,N′-bis(trimethylsilyl)-urea. Among the trialkylsilyl halides, trialkylsilylchloride is preferred, more preferred trialkylsilylchloride being trimethylsilyl chloride and triethylsilyl chloride.

Preferable solvents used in silylation step are selected from esters such as ethyl acetate, methyl acetate, isopropyl acetate, tert-butyl methyl acetate and ethyl formate; acetonitrile; dimethylsulfoxide; dioxane; cyclohexane; n-hexane; aromatic hydrocarbons such as benzene, toluene, xylene; halogenated hydrocarbons such as methylene chloride, chloroform, carbontetrachloride, ethylene dichloride, etc; ketones such as acetone, methyl ethyl ketone, methyl isobutyl ketone, diethyl ketone etc; ethers such as tert-butyl methyl ether, diethyl ether; diethyl carbonate; and a mixture thereof. More preferable solvents used are methylene chloride, ethyl acetate, cyclohexane, ethylene dichloride

Preferably silylation is carried out by adding the silylating agent such as triethyl silyl chloride or Bis(trimethylsilyl)acetamide to a solution of 1-(1,2-benzisothiazol-3-yl)piperazine of formula II in solvent such as methylene chloride or cyclohexane under stirring for at least about 10 minutes in the presence of a tertiary amine base. Preferable tertiary amine base used is triethylamine, N,N-dimethyl-4-aminopyridine or trimethylamine.

The compounds of formula III are useful intermediates for preparing high purity ziprasidone and pharmaceutically acceptable acid addition salts of ziprasidone; and solvates and hydrates thereof.

Preferable compounds of formula IV used in the reaction in step (b) are the compounds of formula IV wherein X is chloro, bromo or iodo, more preferable compounds are the compounds of formula IV wherein X is chloro.

The preferable solvents used are selected from esters such as ethyl acetate, methyl acetate, isopropyl acetate, tert-butyl methyl acetate and ethyl formate; alcohols such as methanol, ethanol and isopropyl alcohol; acetonitrile; tetrahydrofuran; dimethylformamide; dimethylsulfoxide; dioxane; cyclohexane; n-hexane; aromatic hydrocarbons such as benzene, toluene, xylene, etc.; halogenated hydrocarbons such as methylene chloride, chloroform, carbontetrachloride, ethylene dichloride. etc.; ketones such as acetone, methyl ethyl ketone, methyl isobutyl ketone, diethyl ketone etc.; ethers such as tert-butyl methyl ether, diethyl ether; diethyl carbonate; water or a mixture thereof. More preferable solvents are selected from dimethylformamide, methylisobutylketone, water and a mixture thereof.

The base used to neutralize hydrohalic acid is preferably selected from alkalinemetal carbonates such as, sodium carbonate or potassium carbonate; alkalinemetal bicarbonates such as, sodium bicarbonate or potassium bicarbonate, anhydrous ammonia, aqueous ammonia, pyridine, hydrides and tertiary amines such as, triethylamine or diisopropylethylamine.

The reaction is preferably carried out at 50° C. to reflux temperature of the solvent used, more preferably at 80° C. to reflux temperature of the solvent used and most preferably at reflux temperature of the solvent used.

The reaction may be carried out in the presence of catalytic amount of sodium iodide.

Preferable pharmaceutically acceptable acid addition salt of formula I, but not limited to, are the salts are from succinic acid, maleic acid, tartaric acid, citric acid, cinnamic acid, fumaric acid, hydrochloric acid, hydrobromic acid, hydroiodic acid, methanesulfonic acid and benzenesulfonic acid; more preferable salt being ziprasidone hydrochloride.

The preparation of pharmaceutically acceptable acid addition salts of ziprasidone; and their solvates and hydrates from ziprasidone free base may be performed by conventional methods or by methods known in the prior art.

The intermediates of formula III are novel and forms part of the invention. The compounds of formula III wherein R groups are independently selected from methyl or ethyl are preferable. The compounds of formula III wherein R groups are all methyl or all ethyl are more preferable.

According to another aspect of the present invention there is provided another novel process for preparing ziprasidone and pharmaceutically acceptable acid addition salts of ziprasidone; and solvates and hydrates thereof.

Thus, 1-(1,2-benzisothiazol-3-yl)piperazine of formula II:

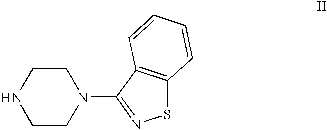

is reacted with 5-(2-haloethyl)-6-chloro-oxindole of formula IV:

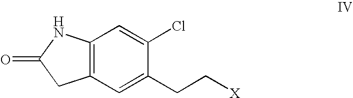

wherein X is fluoro, chloro, bromo or iodo;

in the presence of liquor ammonia and an alkaline metal carbonates such as sodium carbonate or potassium carbonate or an alkaline metal bicarbonate such as sodium bicarbonate or potassium bicarbonate to form ziprasidone of formula I and optionally converted ziprasidone formed into a pharmaceutically acceptable acid addition salts of ziprasidone; or a solvate or a hydrate thereof.

Preferably compounds of formula IV used are those wherein X is chloro, bromo or iodo, more preferable being chloro.

According to another aspect of the present invention there is provided still another novel process for preparing ziprasidone and pharmaceutically acceptable acid addition salts of ziprasidone; and solvates and hydrates thereof.

Thus, 1-(1,2-benzisothiazol-3-yl)piperazine of formula II:

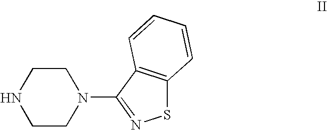

is reacted with 5-(2-haloethyl)-6-chloro-oxindole of formula IV:

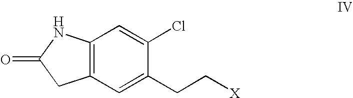

wherein X is fluoro, chloro, bromo or iodo;
   in the presence of pyridine and aqueous monomethylamine to form ziprasidone of formula I and optionally converted ziprasidone formed into a pharmaceutically acceptable acid addition salts of ziprasidone; or a solvate or a hydrate thereof.

Preferable compounds of formula IV used are those wherein X is chloro, bromo or iodo, more preferable being chloro.

According to another aspect of the present invention, there is provided a novel purification method for ziprasidone and pharmaceutically acceptable acid addition salts of ziprasidone; and solvates and hydrates thereof via the formation of novel intermediates.

It is known that ziprasidone free base is insoluble or less soluble in common and commercially used solvents. Therefore, purification of ziprasidone free base is not readily possible by common purification methods such as recrystallization from its solution.

It is also known that commonly used acid addition salts of ziprasidone such as hydrohalide salts are insoluble or less soluble in common and commercially used solvents. Therefore, purification of acid addition salts of ziprasidone are not readily possible by common purification methods such as recrystallization from their solutions.

Ziprasidone and pharmaceutically acceptable acid addition salts of ziprasidone; and solvates and hydrates thereof can be obtained in desired particle size distribution by compacting corresponding crystalline solids by suitable means.

Particle size distribution of ziprasidone, pharmaceutically acceptable acid addition salts of ziprasidone; and solvates and hydrates thereof can be controlled by a suitable compacting method using a compacting machine. Thus, for example, by using this method the said ziprasidone acid addition salts or hydrates can be obtained with mean particle size of about 80 microns or above.

The present invention provides a novel process for purification of ziprasidone free base or a pharmaceutically acceptable acid addition salts of ziprasidone; or a solvate or a hydrate, the said process comprises:

i) silylating crude ziprasidone of formula I:

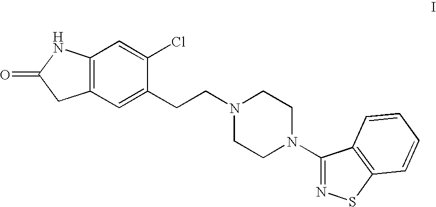

with a silylating agent to form silyl compound of formula V:

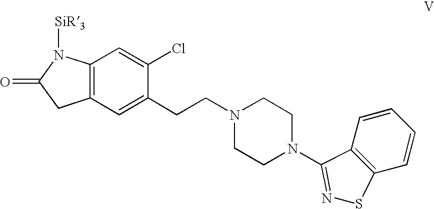

wherein R′ groups are independently alkyl, and

ii) deblocking the silyl protecting group of the compound of formula V formed in step (i) to precipitate ziprasidone of formula I as ziprasidone free base or a pharmaceutically acceptable acid addition salt; or a solvate or a hydrate thereof, as crystalline salt.

The crude ziprasidone refers to ziprasidone for which purification is desired. Usually the purification, according to the novel purification method, crude ziprasidone yields ziprasidone in high performance liquid chromatographic (HPLC) purity above about 94% and typically in above about 98% purity.

Crude ziprasidone may be obtained from a process described in the prior art. Crude ziprasidone may also be prepared from impure acid addition salts of ziprasidone; or their solvates or hydrates by neutralizing with a base and isolating ziprasidone free base from the reaction mass.

Silylation can be performed by conventional method using conventional silylating agents.

Preferable silylating agents are selected from trialkylsilyl halides, N,O-bis(trimethylsilyl)-acetamide and N,N′-bis(trimethylsilyl)-urea. Among the trialkylsilyl halides, trialkylsilylchloride is preferred, more preferred trialkylsilyl chloride being trimethylsilyl chloride and triethylsilyl chloride.

Preferable solvents used in silylation step are selected from esters such as ethyl acetate, methyl acetate, isopropyl acetate, tert-butyl methyl acetate and ethyl formate; acetonitrile; dimethylsulfoxide; dioxane; aromatic hydrocarbons such as benzene, toluene, xylene, halogenated hydrocarbons such as methylene chloride, chloroform, carbontetrachloride, ethylene dichloride, etc; ketones such as acetone, methyl ethyl ketone, methyl isobutyl ketone, diethyl ketone etc; ethers such as tert-butyl methyl ether, diethyl ether; diethyl carbonate; more preferable solvents used are methylene chloride, ethyl acetate, cyclohexane, ethylene dichloride; and a mixture thereof.

Preferably silylation is carried out by adding the silylating agent such as triethyl silyl chloride, Bis(trimethylsilyl)acetamide or N,N′-(bis(trimethylsilyl)-urea to a solution of ziprasidone of formula I in an aprotic solvent such as methylene chloride or cyclohexane under stirring for at least about 10 minutes in the presence of a tertiary amine base. Preferable tertiary amine base used is triethylamine, N,N-dimethyl-4-aminopyridine or trimethylamine.

Deblocking of the compounds of the formula V may be performed by the processes known for deblocking of N-silylprotecting groups.

Preferably deblocking can be performed by contacting the silyl compound of formula V with a protic solvent, water or an acid for sufficient time to effect deblocking.

The silyl compound of formula V may be in a solution or in isolated form before contacting with the said protic solvent, water or the acid.

The choice of the protic solvent is not critical and preferably selected from alcohols.

Deblocking step is usually associated with precipitation of ziprasidone as crystalline solid.

Silyl compounds of formula V are novel and forms part of the invention. Preferred compounds of formula V are compounds of formula V wherein R′ groups are independently methyl or ethyl more preferred compounds are those wherein R′ groups are all methyl or all ethyl.

As a preferred process, pharmaceutically acceptable acid addition salts; or solvates or hydrates thereof can directly be crystallized by using corresponding hydrohalic acid such as hydrochloric acid for deblocking.

The compounds of formulas II and IV used in the processes of the present invention are known and may be prepared by the processes described in the art.

Preferable pharmaceutically acceptable acid addition salts of ziprasidone but not limited to those forms are succinic acid, maleic acid, tartaric acid, citric acid, cinnamic acid, fumaric acid, hydrochloric acid, hydrobromic acid, hydroiodic acid and benzenesulfonic acid.

‘Alkyl’ refers branched or straight C1-C4-alkyl group.

The invention will now be further described by the following examples, which are illustrative rather than limiting.

### Example 1

Methylene chloride (200 ml) is added to 1-(1,2-Benzisothiazol-3-yl)piperazine (15 gm), stirred for 10 minutes and then triethylamine (22 ml) is added drop wise for 10 minutes. Trimethylsilyl chloride (15 ml) is added drop wise to the reaction mass for 20 minutes, tetrabutyl ammonium bromide (5 gm) is added and stirred for 1 hour at 25-30° C. Then the reaction mass is heated to 40° C. and methylene chloride is distilled under vacuum. To this reaction mass sodium carbonate (16 gm) and 5-(2-chloroethyl)-6-chloro-oxindole (16 gm) are added, stirred for 10 minutes and added water (400 ml) and sodium iodide (2 gm). The contents are heated to 10° C. and stirred for 7 hours 30 minutes at 95-100° C. Solid is filtered and slurried in 200 ml of water, filtered and washed with water (100 ml). Then the solid is slurried in isopropyl alcohol (75 ml) at reflux, refluxed for 1 hour and then filtered the solid at reflux point to give 15 gm of ziprasidone (HPLC purity: 98.36%).

### Example 2

The mixture of methylene chloride (130 ml) and 1-(1,2-Benzisothiazol-3-yl)piperazine (9.5 gm) is stirred for 10 minutes, triethylamine (17 ml) is added drop wise for 15 minutes at 25-30° C. and then tetrabutyl ammonium bromide (4 gm) is added. Then trimethylsilyl chloride (9.5 ml) is added to the contents drop wise for 20 minutes and stirred for 1 hour. The reaction mass is heated to 40° C. and methylene chloride is distilled off under vacuum. Then the reaction mass is cooled to 30° C., dimethylformamide (75 ml) is added and stirred for 15 minutes. The reaction mass is filtered on hi-flo and washed with 50 ml of dimethyl formamide.

The mixture of 5-(2-Chloroethyl)-6-chloro-oxindole (10 gm), water (25 ml), dimethyl formamide (35 ml) and sodium carbonate (10 gm) is heated to 110° C. and to this mixture, above filtrate is slowly added drop wise at same temperature for 30 minutes. Then the reaction mass is stirred until completion of the reaction and then cooled to 30° C. The reaction mass is added to chilled water (500 ml) and stirred for 20 minutes. The solid is filtered, slurried in isopropyl alcohol (200 ml). Then the solid is filtered, washed with isopropyl alcohol (100 ml), the solid is again slurried in isopropyl alcohol at reflux and refluxed for 1 hour. Then resulting solid is filtered at reflux point and washed with isopropyl alcohol (60 ml) to give 10 gm of ziprasidone (HPLC purity: 99.05%).

### Example 3

Ziprasidone base (70 gm) is dissolved in methanol (700 ml), cooled to 10° C. and then methanolic hydrochloric acid solution (15%, 70 ml) is added for 20 minutes at 10-15° C. The contents are stirred for 1 hour at 10-15° C., filtered the solid and dried at 65-70° C. for 7 hours to give 70 gm of pure anhydrous ziprasidone hydrochloride (HPLC purity: 99.2%).

### Example 4

Ziprasidone base (70 gm) is dissolved in methanol (700 ml), cooled to 10° C. and then concentrated hydrochloric acid solution (15%, 150 ml) is added for 20 minutes at 10-15° C. The contents are stirred for 1 hour at 10-15° C., filtered the solid and dried at 65-70° C. for 7 hours to give 67 gm of pure ziprasidone hydrochloride hemihydrate (HPLC purity: 99.7%). The ziprasidone hydrochloride hemihydrate, thus obtained, is subjected to compacting in compactor for 8 hours to obtain ziprasidone hydrochloride hemihydrate with mean particle size of 110 microns.

### Example 5

Water (180 ml) is added to ziprasidone free base (12 gm) and then concentrate hydrochloric acid is added at 25-35° C. under stirring. The temperature of reaction mixture is raised to 60-65° C. and heated for 3-4 hours at 60-65° C. The contents are filtered and the solid is washed with water, slurried in acetone to obtain 12 gm of ziprasidone hydrochloride monohydrate (HPLC purity: 99.4%).

### Example 6

Crude ziprasidone (5 gm, HPLC purity: 91%) methylene chloride (50 ml) and triethylamine (4 ml) are stirred for 10 minutes at 25-30° C. and then dimethylaminopyridine (50 mg) is added. To the above mixture trimethylsilyl chloride (3 ml) is added slowly for 10 minutes and maintained at 25-30° C. for 1 hour 30 minutes. The contents are subjected to carbon treatment and then filtered on hi-flo and washed with methylene chloride. To this filtrate is added isopropyl alcohol (100 ml), heated at 60-65° C. for 1 hour 30 minutes. The reaction mass is cooled to 25-30° C. and filtered to obtain ziprasidone free base as solid (HPLC purity: 99.4%).

### Example 7

Crude ziprasidone (5 gm, HPLC purity: 93.2%), methylene chloride (50 ml) and triethylamine (4 ml) are stirred for 10 minutes at 25-30° C. and then dimethylaminopyridine (50 mg) is added. To the above mixture trimethylsilyl chloride (3 ml) is added slowly for 10 minutes and maintained at 25-30° C. for 1 hour 30 minutes. The contents are subjected to carbon treatment and then filtered on hi-flo and washed with methylene chloride. To this filtrate is added aqueous hydrochloric acid solution (5 ml conc. HCl+75 ml of water) and then stirred for 10 minutes. The separated solid is filtered and washed with water and dried to obtain ziprasidone hydrochloride (HPLC purity: 99.6%).

### Example 8

1-(1,2-Benzisothiazol-3-yl)piperazine (14 gm) and 5-(2-haloethyl)-6-chloro-oxindole (13.5 gm) is added to the mixture of pyridine (100 ml) and aqueous monomethylamine (40%, 100 ml), heated up to 80° C. and maintained for 10 hours. After usual work up 12 gm of ziprasidone (HPLC purity of 99.1%) is obtained.

### Example 9

1-(1,2-Benzisothiazol-3-yl)piperazine (14 gm) and 5-(2-haloethyl)-6-chloro-oxindole (13.5 gm) is added to the mixture of liquor ammonia (200 ml) and potassium carbonate (20 gm), heated to 80° C. and maintained for 12 hours followed by usual work up to give ziprasidone (12 gm) as crystalline solid (HPLC purity of 99.4%).

1. A process for preparing 5-[2-[4-(1,2-benzisothiazol-3-yl)-1-piperazinyl]ethyl]-6-chloro-1,3-dihydro-2H-indol-2-one (ziprasidone) of the formula I:
Ziprasidone of formula (I): 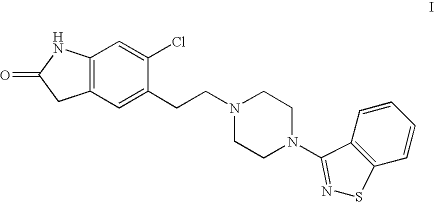
or a pharmaceutically acceptable salt thereof; or a solvate or a hydrate thereof; which comprises:
a) silylating 1-(1,2-benzisothiazol-3-yl)piperazine of the formula II: 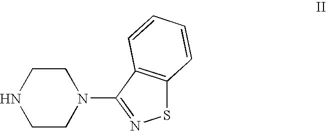
with a silylating agent to form a compound of the formula III: 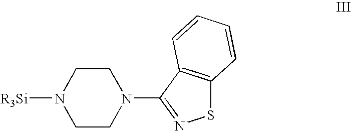
wherein R is independently alkyl;
b) reacting the silyl compound of the formula III with a 5-(2-haloethyl)-6-chloro-oxindole compound of the formula IV: 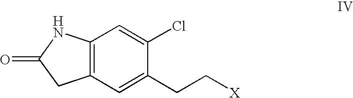
wherein X is fluoro, chloro, bromo or iodo;
in a solvent in the presence of a base to neutralize the hydrohalic acid, at about 40° C. to the reflux temperature of the solvent used to form the compound of formula I and optionally converting the compound of formula I into a pharmaceutically acceptable acid addition salt thereof, or a solvate or a hydrate thereof.

2. The process according to claim 1 wherein the silylation step (a) is carried out with a silylating agent in the presence of a solvent and a tertiary amine base.

3. The process according to claim 2 wherein the silylating agent is selected from trialkylsilyl halides, N,O-bis(trimethylsilyl)-acetamide and N,N′-bis(trimethylsilyl)-urea.

4. The process according to claim 3 wherein the silylating agent is selected from trialkylsilyl halides.

5. The process according to claim 4 wherein the silylating agent is a trialkyl silyl chloride.

6. The process according to claim 3 wherein the silylating agent is selected from trimethylsilyl chloride, triethylsilyl chloride, N,O-bis(trimethylsilyl)-acetamide and N,N′-bis(trimethylsilyl)-urea.

7. The process according to claim 6 wherein the silylating agent is trimethylsilyl chloride.

8. The process according to claim 1 wherein the solvent used in the silylating step (a) is selected from ethyl acetate, methyl acetate, isopropyl acetate, tert-butyl methyl acetate, ethyl formate, acetonitrile, dimethylsulfoxide, dioxane, cyclohexane, n-hexane, benzene, toluene, xylene, methylene chloride, chloroform, carbontetrachloride, ethylene dichloride, acetone, methyl ethyl ketone, methyl isobutyl ketone, diethyl ketone, tert-butyl methyl ether, diethyl ether, diethyl carbonate, and a mixture thereof.

9. The process according to claim 8 wherein the solvent is selected from methylene chloride, ethylacetate, cyclohexane, ethylene dichloride and a mixture thereof.

10. The process according to claim 9 wherein the solvent is methylene chloride.

11. The process according to claim 1 wherein X of the compound of formula IV is chloro, bromo or fluoro.

12. The process according to claim 11 wherein X is chloro.

13. The process according to claim 1 wherein the solvent used in step (b) is selected from ethyl acetate, methyl acetate, isopropyl acetate, tert-butyl methyl acetate, ethyl formate, methanol, ethanol and isopropyl alcohol, acetonitrile, tetrahydrofuran, dimethylformamide, dimethyl sulfoxide, dioxane, cyclohexane, n-hexane, benzene, toluene, xylene, methylene chloride, chloroform, carbontetrachloride, ethylene dichloride, acetone, methyl ethyl ketone, methyl isovutyl ketone, diethyl ketone, tert-butyl methyl ether, diethyl ether, diethyl carbonate, water and a mixture thereof.

14. The process according to claim 13 wherein the solvent is selected from dimethylformamide, methylisobutylketone, water or a mixture thereof.

15. The process according to claim 1 wherein the base used to neutralize hydrochloric acid is selected from alkaline metal carbonates, alkaline metal bicarbonates, anhydrous ammonia, aqueous ammonia, pyridine, hydrides and tertiary amines.

16. The process according to claim 15 wherein the base is selected from sodium carbonate, potassium carbonate, sodium bicarbonate, potassium bicarbonate, triethylamine and diisopropylethylamine.

17. The process according claim 16 wherein the base is sodium carbonate or potassium carbonate.

18. The process according to claim 1, wherein the reaction is carried out at about 50° C. to the reflux temperature of the solvent used.

19. The process according to claim 18 wherein the reaction is carried out at about 80° C. to the reflux temperature of the solvent used.

20. The process according to claim 19 wherein the reaction is carried out at the reflux temperature of the solvent used.

21. The process according to claim 17 wherein the base is sodium carbonate.

22. The compounds of the formula III: 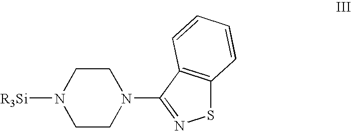
wherein the R₃ groups are independently alkyl.

23. The compound of claim 22 wherein the R groups are independently methyl or ethyl.

24. The compounds of claim 23 wherein the R groups are all methyl or all ethyl.

25. A process for preparing ziprasidone of the formula I 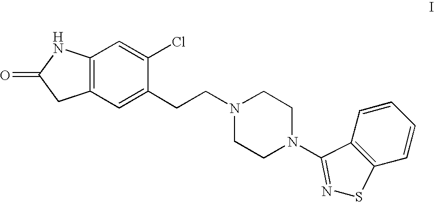
or a pharmaceutically acceptable salt thereof; or a solvate or a hydrate thereof;
which process comprises reacting 1-(1,2-benzisothiazol-3-yl)piperazine of the formula II: 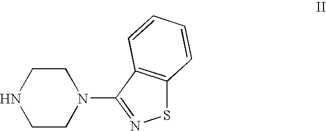
with 5-(2-haloethyl)-6-chloro-oxindole of the formula IV: 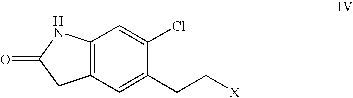
wherein X is fluoro, chloro, bromo or iodo;
in the presence of liquor ammonia and an alkaline metal carbonates or alkaline metal bicarbonate to form ziprasidone of the formula I; and optionally converting the ziprasidone formed into a pharmaceutically acceptable acid addition salts of ziprasidone, or a solvate or a hydrate thereof.

26. The process according to claim 25 wherein X of the formula IV is chloro, bromo or iodo.

27. The process according to claim 26 wherein X is Cl.

28. A process according to claim 1 further comprising controlling the mean particle size of ziprasidone, pharmaceutically acceptable acid addition salts of ziprasidone, and solvates and hydrates thereof formed in step (b) by a method of compacting using a compacting machine.

29. The process according to claim 28 wherein the pharmaceutically acceptable acid addition salt is ziprasidone hydrochloride.

30. The process according to claim 29 wherein the mean particle size of the product is about 80 microns or above.

31. A process for preparing ziprasidone of the formula I 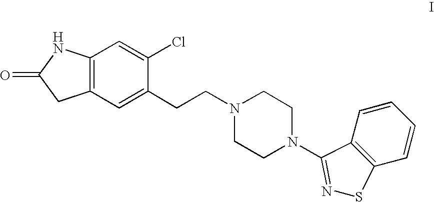
or a pharmaceutically acceptable salt thereof, or a solvate or a hydrate thereof,
which process comprises reacting 1-(1,2-benzisothiazol-3-yl)piperazine of the formula II: 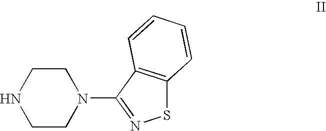
with 5-(2-haloethyl)-6-chloro-oxindole of formula IV: 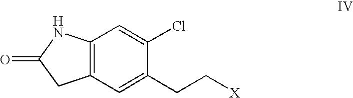
wherein X is fluoro, chloro, bromo or iodo;
in the presence of pyridine and aqueous monomethylamine to form ziprasidone of the formula I and optionally converting the ziprasidone formed into a pharmaceutically acceptable acid addition salts of ziprasidone, or a solvate or a hydrate thereof.

32. The process according to claim 31 wherein X of the formula IV is chloro, bromo or iodo.

33. The process according to claim 32 wherein X is chloro or bromo.

34. The process according to claim 33 wherein X is chloro.

35. The process according to claim 31 wherein the pharmaceutically acceptable salt is ziprasidone hydrochloride.

36. The process according to claim 31 wherein the hydrate is ziprasidone hydrochloride hemihydrate.

37. A process for purifying ziprasidone free base or a pharmaceutically acceptable acid addition salts of ziprasidone, or a solvate or a hydrate, the process comprising:
i) silylating crude ziprasidone of the formula I: 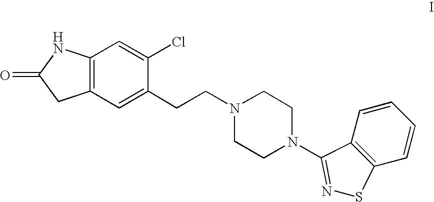
with a silylating agent to form a silyl compound of the formula V: 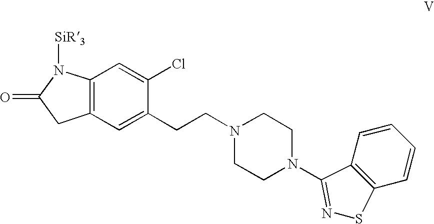
wherein R′ groups are independently alkyl, and
ii) deblocking the silyl protecting group of the compound of the formula V formed in step (i) to precipitate ziprasidone of the formula I as ziprasidone free base or a pharmaceutically acceptable acid addition salt, or a solvate or a hydrate thereof as a crystalline salt.

38. The process according to claim 37 wherein the silylating agent is selected from trialkylsilyl halides, N,O-bis(trimethylsilyl)-acetamide and N,N′-bis(trimethylsilyl)-urea.

39. The process according to claim 38 wherein the trialkylsilyl halide is trialkylsilyl chloride.

40. The process according to claim 38 wherein the silylating agent is selected from trimethylsilyl chloride, triethylsilyl chloride, N,O-bis(trimethylsilyl)-acetamide and N,N-bis(trimethylsilyl)-urea.

41. The process according to claim 40 wherein the silylating agent is trimethyl silyl chloride.

42. The process according to claim 37 wherein the solvent used in the silylation step is selected from ethyl acetate, methyl acetate, isopropyl acetate, tert-butyl methyl acetate, ethyl formate, acetonitrile, dimethylsulfoxide, dioxane, benzene, toluene, xylene, methylene chloride, chloroform, carbontetrachloride, ethylene dichloride, acetone, methyl ethyl ketone, methyl isobutyl ketone, diethyl ketone, tert-butyl methyl ether, diethyl ether, diethyl carbonate and a mixture thereof.

43. The process according to claim 42 wherein the solvent used is selected from methylene chloride, ethylacetate, cyclohexane, ethylene dichloride, toluene, carbon tetrachloride and a mixture thereof.

44. The process according to claim 43 wherein the solvent is selected from methylene chloride, ethyl acetate, cyclohexane, ethylene dichloride and a mixture thereof.

45. The process according to claim 37 wherein the silylation is carried out in the presence of a tertiary amine base.

46. The process according to claim 45 wherein the base is triethylamine, N,N-dimethyl-4-aminopyridine or trimethylamine.

47. The process according to claim 37 wherein the deblocking step (ii) is carried out by contacting the silyl compound of the formula V with a protic solvent, water or an acid for sufficient time to effect deblocking.

48. The process according to claim 47 wherein the protic solvent is an alcohol, and the acid is selected from hydrochloric acid, hydrobromic acid, hydroiodic acid and methanesulfonic acid.

49. The process according to claim 48 wherein the alcohol is ethanol or methanol.

50. The process according to claim 48 wherein the acid is hydrochloric acid.

51. The process according to claim 50 wherein ziprasidone is isolated as ziprasidone hydrochloride or hydrates thereof.

52. The process according to claim 51 wherein the hydrates of ziprasidone hydrochloride are ziprasidone hydrochloride hemihydrate or ziprasidone hydrochloride monohydrate.

53. The process according to claim 52 wherein the hydrate of ziprasidone hydrochloride is ziprasidone hydrochloride hemihydrate.

54. The process according to claim 48 wherein the protic solvent is methanol.

55. The process according to claim 47 wherein the solvent is water.

56. Compounds of the formula V: 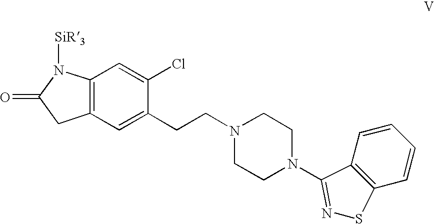
wherein the R¹ groups are independently alkyl.

57. The compounds as defined in claim 56 wherein the R¹₃ groups is independently methyl or ethyl.

58. The compounds as defined in claim 57, wherein the R¹₃ group is methyl or all ethyl.

59. The process according to claim 11 wherein the solvent used in step (b) is selected from ethyl acetate, methyl acetate, isopropyl acetate, tert-butyl methyl acetate, ethyl formate, methanol, ethanol and isopropyl alcohol, acetonitrile, tetrahydrofuran, dimethylformamide, dimethyl sulfoxide, dioxane, cyclohexane, n-hexane, benzene, toluene, xylene, methylene chloride, chloroform, carbontetrachloride, ethylene dichloride, acetone, methyl ethyl ketone, methyl isovutyl ketone, diethyl ketone, tert-butyl methyl ether, diethyl ether, diethyl carbonate, water and a mixture thereof.

60. The process according to claim 12 wherein the solvent used in step (b) is selected from ethyl acetate, methyl acetate, isopropyl acetate, tert-butyl methyl acetate, ethyl formate, methanol, ethanol and isopropyl alcohol, acetonitrile, tetrahydrofuran, dimethylformamide, dimethyl sulfoxide, dioxane, cyclohexane, n-hexane, benzene, toluene, xylene, methylene chloride, chloroform, carbontetrachloride, ethylene dichloride, acetone, methyl ethyl ketone, methyl isovutyl ketone, diethyl ketone, tert-butyl methyl ether, diethyl ether, diethyl carbonate, water and a mixture thereof.

61. The process according to claim 59 wherein the solvent is selected from dimethylformamide, methylisobutylketone, water or a mixture thereof.

62. The process according to claim 60 wherein the solvent is selected from dimethylformamide, methylisobutylketone, water or a mixture thereof.

63. The process according to claim 28 wherein the pharmaceutically acceptable acid addition salt is ziprasidone hydrochloride and the hydrate is ziprasidone hydrochloride hemihydrate.

64. The process according to claim 63 wherein the mean particle size of the product is about 80 microns or above.

65. The process according to claim 28 wherein the pharmaceutically acceptable acid addition salt is ziprasidone hydrochloride and the hydrate is ziprasidone hydrochloride monohydrate.

66. The process according to claim 65 wherein the mean particle size of the product is about 80 microns or above.

67. A pharmaceutical composition comprising ziprasidone hydrochloride hemihydrate and ziprasidone hydrochloride monohydrate.
